(19) United States
(12) Patent Application Publication
(10) Pub. No.: US 2020/0190046 A1
(43) Pub. Date: Jun. 18, 2020

(54) NOVEL ESTERIFICATION CATALYST AND USES THEREOF

(71) Applicant: Archer Daniels Midland Company, Decatur, IL (US)

(72) Inventors: Erik Hagberg, Decatur, IL (US); Andrew J. Ingram, Champaign, IL (US); Erin J. Rockafellow, Decatur, IL (US); Jaime Sullivan, Fitchburg, WI (US); Kenneth F. Stensrud, Decatur, IL (US)

(73) Assignee: Archer Daniels Midland Company, Decatur, IL (US)

(21) Appl. No.: 16/500,283

(22) PCT Filed: Mar. 20, 2018
(86) PCT No.: PCT/US18/23354

§ 371 Date: Oct. 2, 2019

Related U.S. Application Data
(60) Provisional application No. 62/481,802, filed on Apr. 5, 2017.

(51) Int. Cl. C07D 307/68; B01J 31/12

(52) U.S. Cl. CPC C07D 307/68; B01J 31/122

(57) ABSTRACT

Tin (II) glucarate is found to be effective alone and in combination with other tin compounds for catalyzing the reaction of carboxylic acids such as furan-2,5-dicarboxylic acid, terephthalic acid and adipic acid with alcohols such as the C1-C3 alcohols.

### TECHNICAL FIELD

The present invention relates generally to processes for making the esters of carboxylic acids and to the catalysts useful therein, and in particular aspects, to the esterification of 2,5-furandicarboxylic acid, terephthalic acid and adipic acid and the catalysts useful therein.

### BACKGROUND ART

In recent years, an increasing effort has been devoted to identifying new and effective ways to use renewable feedstocks for the production of organic chemicals. The production of furans and furan derivatives from six-carboned carbohydrates has been an area of particular interest, with 2,5-furandicarboxylic acid (or FDCA) being an example as a promising green alternative to terephthalic acid.

The use of FDCA as a replacement for terephthalic acid or as a monomer in general, however, poses a number of challenges, in that FDCA has very limited solubility in many common organic solvents and further is characterized by an extremely high melting point (>300° C.)). Simple chemical modifications, such as esterification, often enable one to overcome these difficulties. Esterification of FDCA with methanol to make dimethyl 2,5-furandicarboxylate (FDME), for example, provides a material with a melting point of 112° C. and a boiling point of 140-145° C. (10 torr), and which can be solubilized in a number of common organic solvents.

Over the years, esterification of FDCA by autocatalysis has been demonstrated by several research groups in many publications, but high temperatures and long reaction times are required to attain commercially viable conversions of FDCA and yields of the targeted esters, adding significantly to the cost of manufacture on an industrial or commercial scale operation. Brønsted acid catalysis enables improved FDCA conversions and higher ester yields for a given temperature and reaction time, but also readily drives alcohol condensation to undesired, low molecular weight ethers, reducing the overall yield of the desired FDME product and introducing difficulties associated with the removal of these ethers to the degree necessary to attain the high levels of purity which are typically needed for a subsequent polymerization method, using FDME as a monomer. Various Lewis acid catalysts have also been considered, but these often suffer from limited activity and from a propensity to generate undesired Brønsted acids in the presence of water.

### SUMMARY OF THE INVENTION

The following presents a simplified summary of the invention in order to provide a basic understanding of some of its aspects. This summary is not an extensive overview of the invention, thus the mention or omission of a particular feature should not be understood as implying, respectively, that the feature is indispensable or of lesser significance. The sole purpose of this summary is to present some concepts of the invention in a simplified form as a prelude to the more detailed description that is presented later.

With this understanding, the present invention in one aspect relates to a novel catalyst composition comprising tin (II) glucarate.

In certain embodiments, the tin glucarate is combined with one or more other tin compounds selected from the group consisting of the tin (II) salts and those organotin (tin (IV)) catalysts described in commonly assigned WO 2017/091437.

In certain embodiments, tin glucarate is combined with one or more of tin acetate, tin octoate, tin chloride and tin oxalate.

In other embodiments, tin glucarate is combined with one or more of butylstannoic acid (BSA), dibutyltin oxide (DBTO), dibutyltin diacetate, butyltin tris 2-ethylhexanoate, dibutyltin maleate, dibutyltin dilaurate, dioctyltin oxide, dibutyltin bis(1-thioglyceride), dibutyltin dichloride, and monobutyltin dihydroxychloride.

In other embodiments, the novel catalyst composition consists essentially of tin glucarate, and in still another embodiment, the novel catalyst composition consists simply of tin glucarate with no other esterification catalysts.

In another aspect, the present invention relates to the use of a catalyst composition of the present invention in an esterification reaction.

In one embodiment, a catalyst composition of the present invention is used for the esterification of furan-2,5-dicarboxylic acid with an alcohol, particularly but without limitation thereto, a C₁-C₃ alcohol.

In another embodiment, a catalyst composition of the present invention is used for the esterification of terephthalic acid.

In yet another embodiment, a catalyst composition of the present invention is used for the esterification of adipic acid.

### DETAILED DESCRIPTION OF EMBODIMENTS

As used in this application, the singular forms “a”, “an” and “the” include plural references unless the context clearly indicates otherwise. The term “comprising” and its derivatives, as used herein, are similarly intended to be open ended terms that specify the presence of the stated features, elements, components, groups, integers, and/or steps, but do not exclude the presence of other unstated features, elements, components, groups, integers and/or steps. This understanding also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers, and/or steps. The term “consisting essentially of”, as used herein, is intended to specify the presence of the stated features, elements, components, groups, integers, and/or steps, as well as those that do not materially affect the basic and novel characteristic(s) of stated features, elements, components, groups, integers, and/or steps.

Unless otherwise indicated, any definitions or embodiments described in this or in other sections are intended to be applicable to all embodiments and aspects of the subjects herein described for which they would be suitable according to the understanding of a person of ordinary skill in the art.

As previously indicated, the present invention relates in a first aspect to a novel catalyst composition comprising tin (II) glucarate, which has proven an effective esterification catalyst.

Glucarate salts are mentioned in the art for a few different applications: GB 836.979 describes the use of potassium or sodium saccharate (glucarate) salts as currency efficiency improver additives in baths for the electrodeposition of copper and copper alloys; U.S. Pat. No. 4,946,668 to Daddona et al. described the use of a complex of technetium-99m and glucarate as an imaging agent for the study, detection or diagnosis of tumors; US 2012/0295986 to Smith et al. describes calcium sequestering compositions comprised of potassium, calcium, sodium, zinc, ammonium and lithium salts of glucaric acid with aluminum salts; and U.S. Pat. No. 7,655,678 to Gupta et al. describes pharmaceutical compositions for the management of tumors including calcium glucarate salts. The sole apparent mention of glucarate salts for a catalytic use is in a Czech patent application, CS 122217, from 1967, wherein alkali metal saccharates are described as effective catalysts for the reaction of sucrose and fatty acid esters, e.g., methyl palmitate. Potassium, sodium and lithium salts are mentioned specifically, and there is no mention or suggestion of catalytic utility of any other salt of saccharic (or glucaric) acid for the proposed transformation or any other transformation. It is presumed that the salts in question were prepared by reaction of saccharic acid with the elemental alkali earth metals, a methodology those skilled in the art would recognize as unsuited to the preparation, for example, of tin glucarate.

In certain embodiments of the present invention according to this first aspect, a catalyst composition is contemplated wherein the tin glucarate is combined with one or more other tin compounds selected from the group consisting of the tin (II) salts and those organotin (tin (IV)) catalysts described in commonly assigned, copending Patent Cooperation Treaty Application Serial Number PCT/US2016/62491, filed Nov. 17, 2016 for “Organotin Catalysts in Esterification Processes of Furan-2,5-Dicarboxylic Acid (FDCA)” and claiming the benefit of U.S. Provisional Application No. 62/259,124, filed Nov. 24, 2015. Preferred tin (II) salts include tin acetate, tin octoate, tin chloride and tin oxalate, while preferred organotin catalysts include butylstannoic acid (BSA), dibutyltin oxide (DBTO), dibutyltin diacetate, butyltin tris 2-ethylhexanoate, dibutyltin maleate, dibutyltin dilaurate, dioctyltin oxide, dibutyltin bis(1-thioglyceride), dibutyltin dichloride, and monobutyltin dihydroxychloride.

As demonstrated by the examples following, in using tin glucarate in combination with other tin catalysts for the making of commercially important esters by the combination of FDCA, adipic acid or terephthalic acid with alcohols, at least equivalent and sometimes greater yields can be achieved as compared with the use of the other, perhaps more costly or less easily procured tin catalysts alone.

The present invention generally contemplates combinations of tin glucarate in any proportion with other tin (II) salts or organotin catalysts, but because of its comparatively low cost, it is expected that it will be preferred that tin glucarate comprise at least 80 percent by weight, more preferably at least 85 percent by weight, still more preferably at least 90 percent by weight of the total weight of tin compounds in the composition.

In other embodiments, the novel catalyst composition will consist essentially of tin glucarate, and in still another embodiment, the novel catalyst composition may consist entirely of tin glucarate with no other tin compounds being present.

In a second aspect, the present invention relates to the use of a catalyst composition of the present invention in an esterification reaction. In particular embodiments, tin glucarate or a tin glucarate-containing catalyst composition is used for catalyzing the reaction of a carboxylic acid such as, but not being limited to, FDCA, adipic acid or terepththalic acid with an alcohol, with preferred alcohols being selected from the group of C₁-C₃ alcohols. A preferred application is in the esterification of FDCA, particularly for forming the diesters of FDCA with the C₁-C₃ alcohols, especially the dimethyl ester FDME.

A suitable tin glucarate catalyst may be made by dissolving potassium glucarate in water and also dissolving tin (II) chloride in water, then mixing the two solutions together and adjusting the pH to from 6 to 7, whereupon the tin glucarate will precipitate out and be recoverable by filtration.

The present invention is more particularly illustrated by the following, non-limiting examples:

### Example 1 and Comparative Examples 1-3

A 75 cc Parr autoclave equipped with a glass enclosed magnetic stir abr was charged with 6 grams of FDCA, 60 mg of tin glucarate and 30 g of methanol, The vessel was sealed, then the contents were heated over thirty minutes from ambient temperature to a temperature of 200 degrees Celsius with continuous agitation at 875 rpm. After an hour at 200 degrees, the vessel was flash cooled in an ice bath, and on reaching 25 degrees Celsius the contents of the vessel were removed. The residual paste found therein was dissolved in tetrahydrofuran, dried under reduced pressure, and then analyzed by UPLC-UV. More than 99 percent by weight of the FDCA was found to have been converted, and the product included 85 weight percent of the dimethyl ester of FDCA (FDME), with the balance being the monomethyl ester (FDMME). The experiment was repeated using the same apparatus, procedure and conditions with other tin (II) catalysts, namely, stannous octoate, stannous chloride and stannous oxalate. The conversions of FDCA and yields of FDME realized with these catalysts were: stannous octoate, 96 percent by weight of FDCA converted, yielding a product containing 69 percent by weight of FDME; stannous chloride, 94 percent of FDCA converted, with a product containing 81 percent of FDME; and stannous oxalate, 97 percent of FDCA converted to product of which 78 percent was FDME.

### Example 2 and Comparative Examples 4 and 5

In the same Parr reactor setup as used for Example 1 and Comparative Examples 1-3, 17 weight percent of terephthalic acid in methanol was combined with 0.5 mole percent of each of tin glucarate, tin acetate and tin octoate. After heating over a period of thirty minutes to a reactor temperature of 200 degrees Celsius and maintaining this temperature for thirty minutes under constant stirring, the Parr reactor was flash cooled to 25 degrees Celsius in an ice bath, then the residual paste was withdrawn, dissolved in THF, dried under reduced pressure and analyzed by UPLC-UV. The results are shown in Table 1 as follows, wherein residual unconverted terephthalic acid (TPA) and the monomethyl terephthalate (MMT) and dimethyl terephthalate (DMT) are shown for each catalyst:

TABLE 1

| Catalyst | TPA (wt %) | MMT (wt %) | DMT (wt %) |
|---|---|---|---|
| Tin acetate | 0.4 | 10.7 | 88.9 |
| Tin glucarate | 0.4 | 13.1 | 86.4 |
| Tin octoate | 3.8 | 36.3 | 59.9 |

### Example 3 and Comparative Examples 6-7

In the same Parr reactor setup as used for Example 1 and Comparative Examples 1-3, 17 weight percent of adipic acid in methanol was combined with 0.5 mole percent of each of tin glucarate, tin acetate and tin octoate. After heating over a period of thirty minutes to a reactor temperature of 200 degrees Celsius and maintaining this temperature for thirty minutes under constant stirring, the Parr reactor was flash cooled to 25 degrees Celsius in an ice bath, then the residual paste was withdrawn, dissolved in THF, dried under reduced pressure and analyzed by nuclear magnetic resonance spectroscopy (¹H NMR) to compare the conversion achieved to the monomethyl and dimethyl adipate esters, without in this instance undertaking to determine how much of the monoesters and diesters were thus made.

The tin glucarate example converted 70 mole percent of the adipic acid to the mono- and diesters, while the tin octoate example converted 69 mole percent to the mono- and diesters and the tin acetate example converted 96 mole percent of the adipic acid to the monoester and diester.

### Example 4

The same Parr reactor setup as used for previous examples was charged with 6 grams of FDCA, 30 grams of methanol, 60 mg of tin glucarate and 6 mg of tin (II) acetate. The vessel was sealed, then heated over a period of thirty minutes to a reaction temperature of 200 degrees Celsius and maintained there with constant magnetic stirring at 875 rpm for one hour. After this time, the vessel was flash cooled in an ice bath to a temperature of 25 degrees Celsius, after which the reactor contents were discharged. The residual paste found therein was dissolved in THF, dried under reduced pressure and then analyzed by UPLC-UV, indicating that more than 99 percent by weight of the FDCA had been converted to 88 weight percent of FDME, with the balance to 100% being the monoester FDMME.

### Example 5

The same Parr reactor setup as used for previous examples was charged with 6 grams of FDCA, 30 grams of methanol, 60 mg of tin glucarate and 6 mg of butylstannoic acid. The vessel was sealed, then heated over a period of thirty minutes to a reaction temperature of 200 degrees Celsius and maintained there with constant magnetic stirring at 875 rpm for one hour. After this time, the vessel was flash cooled in an ice bath to a temperature of 25 degrees Celsius, after which the reactor contents were discharged. The residual paste found therein was dissolved in THF, dried under reduced pressure and then analyzed by UPLC-UV, indicating that more than 99 percent by weight of the FDCA had been converted to 90 weight percent of FDME, with the balance to 100% being the monoester FDMME.

1: Tin (II) glucarate.

2: A mixture of tin (II) glucarate with one or more other tin compounds selected from the group consisting of the tin (II) salts, butylstannoic acid, dibutyltin oxide, dibutyltin diacetate, butyltin tris 2-ethylhexanoate, dibutyltin maleate, dibutyltin dilaurate, dioctyltin oxide, dibutyltin bis(1-thioglyceride), dibutyltin dichloride, and monobutyltin dihydroxychloride.

3: The mixture of claim 2, wherein the tin (II) salts are one or more of tin acetate, tin octoate, tin chloride and tin oxalate.

4: A process for forming an ester of a carboxylic acid and an alcohol, comprising reacting a carboxylic acid with an alcohol in the presence of a catalyst comprising tin (II) glucarate.

5: The process of claim 4, conducted in the presence of a mixture of tin (II) glucarate with one or more other tin compounds selected from the group consisting of the tin (II) salts, butylstannoic acid, dibutyltin oxide, dibutyltin diacetate, butyltin tris 2-ethylhexanoate, dibutyltin maleate, dibutyltin dilaurate, dioctyltin oxide, dibutyltin bis(1-thioglyceride), dibutyltin dichloride, and monobutyltin dihydroxychloride.

6: The process of claim 5, wherein the tin (II) salts are one or more of tin acetate, tin octoate, tin chloride and tin oxalate.

7: The process of claim 5, wherein the tin (II) glucarate is at least 80 percent by weight of the total weight of a tin compound mixture of tin (II) glucarate and one or more other tin compounds combined.

8: The process of claim 7, wherein the tin (II) glucarate is at least 85 percent by weight of the tin compound mixture.

9: The process of claim 8, wherein the tin (II) glucarate is at least 90 percent by weight of the tin compound mixture.

10: The process of claim 4, wherein the carboxylic acid is at least one selected from the group consisting of furan-2,5-dicarboxylic acid, terephthalic acid and adipic acid.

11: The process of claim 10, wherein the alcohol is a C₁-C₃ alcohol.

12: The process of claim 11, wherein furan-2,5-dicarboxylic acid is reacted with methanol to form a methyl ester of furan-2,5-dicarboxylic acid.
